(19) United States
(12) Patent Application Publication
(10) Pub. No.: US 2019/0276791 A1
(43) Pub. Date: Sep. 12, 2019

(54) Cell Culture Sheet

(71) Applicant: Dexerials Corporation, Tokyo (JP)

(72) Inventors: Hirokazu Odagiri, Tokyo (JP); Kazuya Hayashibe, Tokyo (JP); Katsuhiro Doi, Tokyo (JP)

(21) Appl. No.: 16/293,000

(22) Filed: Mar. 5, 2019

(30) Foreign Application Priority Data
Mar. 8, 2018 (JP) 2018-041735

(51) Int. Cl. C12M 1/12; C12M 1/00; C12M 1/22

(52) U.S. Cl. CPC C12M 37/00; C12M 23/10; C12M 23/34

(57) ABSTRACT

A cell culture sheet including: at least one cell culture region contributing to cell culture; and at least one antimicrobial region exhibiting antimicrobial action, which is adjacent to the cell culture region, recessed with respect to the cell culture region, and has a plurality of fine projections that do not exceed a surface of the cell culture region in height.

### CROSS-REFERENCE TO RELATED APPLICATIONS

This application claims priority to Japanese application No. 2018-041735, filed on Mar. 8, 2018 and incorporated herein by reference.

### BACKGROUND OF THE INVENTION

### Field of the Invention

The present invention relates to a cell culture sheet.

### Description of the Related Art

General methods for sterilizing the surface of a cell culture sheet include methods for sterilizing the surface thereof using EOG (ethylene oxide gas) or moist heat (autoclave) and methods for sterilizing the surface thereof with irradiation of gamma-rays or electron beams.

However, there have been problems that gases or residual products generated by EOG adversely affect living organisms, moist heat exposes a subject to a high temperature to cause degeneration, and gamma-rays or electron beams give damage to molecular structures depending on materials to degrade qualities of the materials themselves.

In addition, contamination (inclusion) of microorganisms through the cell culturing process brings about considerably serious influence. It goes without saying that culture needs a sufficiently careful manipulation to avoid contamination. There are taken such approaches that an antibiotic is added to a medium to prevent proliferation of microorganisms if they contaminate the medium in the worst case, an antimicrobial agent is coated on the surface of a culture sheet, and an antimicrobial agent is kneaded with a resin constituting the sheet. However, in order to effectively express the antimicrobial effects, the antibiotics or the antimicrobial agents are necessarily exposed at a high concentration. As a result, there are not only a concern about influence on cells intended to be cultured, but also a risk that resistant microbes are generated by excessively using the above agents.

Meanwhile, antimicrobial sheets using fine structures have conventionally been proposed.

For example, Japanese Patent Application Laid-Open (JP-A) No. 2017-503554 and Japanese Patent No. 5788128 describe that the fine structures projected from the surface of the sheet stick into cell membranes of the microorganisms and the resulting bores fatally affect the microorganisms.

Moreover, Japanese Patent Nos. 5451768 and 6062165 describe that microbes are trapped by concave and convex portions of micron order to inhibit colonization and formation of biofilms.

### SUMMARY OF THE INVENTION

That is, it is generally known that the fine structures physically act on microbes and the like, and are effective in an antimicrobial treatment.

However, when the surfaces of such fine structures are applied to microorganisms, the antimicrobial effects can be obtained, but there is a problem that the surfaces of the fine structures are unsuitable for culturing cells to be cultured on the surfaces of the fine structures, and also kill the cells to be cultured.

The present invention solves the conventionally existing problems and aims to achieve the following object. That is, the object of the present invention is to provide a cell culture sheet that can exhibit an effective antimicrobial action on microorganisms.

Means for solving the above problems are as follows. That is,
<1> A cell culture sheet including:

at least one cell culture region contributing to cell culture; and

at least one antimicrobial region exhibiting antimicrobial action, which is adjacent to the cell culture region, recessed with respect to the cell culture region, and has a plurality of fine projections that do not exceed a surface of the cell culture region in height.
<2> The cell culture sheet according to <1>, wherein the fine projections have a cone shape or a pillar shape.
<3> The cell culture sheet according to <1> or <2>, wherein when the cell culture sheet is seen from a top surface thereof, a plurality of the antimicrobial regions are surrounded by the cell culture region.
<4> The cell culture sheet according to <1> or <2>, wherein when the cell culture sheet is seen from a top surface thereof, a plurality of the cell culture regions are surrounded by the antimicrobial region.
<5> The cell culture sheet according to any one of <1> to <4>, wherein height of the fine projections is 0.7 times or less as high as height from a bottom of the antimicrobial region to the surface of the cell culture region.
<6> The cell culture sheet according to any one of <1> to <5>, wherein depth of the antimicrobial region is 5 μm or more but 100 μm or less.
<7> The cell culture sheet according to any one of <1> to <6>, wherein the cell culture sheet includes a base material and a culture layer on the base material, the culture layer including the cell culture region and the antimicrobial region.

According to the present invention, it is possible to solve the conventionally existing problems, achieve the above-described object, and provide a cell culture sheet that can exhibit an effective antimicrobial action on microorganisms.

### BRIEF DESCRIPTION OF THE DRAWINGS

FIG. 1A is a schematic view (top view) for presenting one example of a cell culture sheet.

FIG. 1B is an enlarged view of the portion circled by the dashed line in FIG. 1A.

FIG. 1C is a cross-sectional view of AA in FIG. 1B.

FIG. 2A is a schematic view (top view) for presenting another example of a cell culture sheet.

FIG. 2B is an enlarged view of the portion enclosed by the dashed line in FIG. 2A.

FIG. 2C is a cross-sectional view of BB in FIG. 2B.

FIG. 3A is a schematic view (top view) for presenting another example of a cell culture sheet.

FIG. 3B is an enlarged view of the portion circled by the dashed line in FIG. 3A.

FIG. 3C is one example of a cross-sectional view of CC in FIG. 3B.

FIG. 3D is another example of the cross-sectional view of CC in FIG. 3B.

FIG. 4A is a schematic view (top view) for presenting a cell culture sheet of Comparative Example 1.

FIG. 4B is a cross-sectional view of AA in FIG. 4A.

### DESCRIPTION OF THE EMBODIMENTS

### (Cell Culture Sheet)

A cell culture sheet of the present invention includes at least one cell culture region and at least one antimicrobial region, and further includes other members if necessary.

The cell culture sheet includes, for example, a base material and a culture layer on the base material, the culture layer including the cell culture region and the antimicrobial region.

In the cell culture sheet, the antimicrobial region is adjacent to the cell culture region, recessed with respect to the cell culture region, and has a plurality of fine projections that do not exceed a surface of the cell culture region in height. As a result, it possible to culture cells intended to be cultured on the cell culture region, and to prevent at least proliferation of microorganisms intended to be antimicrobially treated through antimicrobial action achieved by the antimicrobial region. Therefore, the cell culture sheet can be suitably used for culture of cells that uses neither an antibiotic agent nor an antimicrobial agent.

In the present invention, the term “antimicrobial” means at least one of killing microbes, sterilization, disinfection, antisepsis, and bacteriostasis. The killing microbes solely means killing microorganisms. The sterilization means sterilization or elimination of all microorganisms from the subject object. The disinfection means that specific microorganisms that are pathogenic with respect to humans and animals are killed to prevent infection, and does not mean that all microorganisms are killed. The antisepsis generally means that microorganisms are eliminated from the target subject. The bacteriostasis means inhibition or prevention of proliferation of microorganisms.

The microorganisms refer to, for example, bacteria and fungus.

### <Cell Culture Region>

The cell culture region is a region contributing to cell culture.

The surface of the cell culture contacts with cells.

In the cell culture region, cell walls of the cells are not destroyed depending on its surface shape. In other words, the surface shape of the cell culture region is not particularly limited and may be appropriately selected depending on the intended purpose, so long as it is planar enough not to destroy the cell walls of the cells.

The cells intended to be cultured are not particularly limited and may be appropriately selected depending on the intended purpose, so long as they are cells other than cells of microorganisms intended to be antimicrobially treated (e.g., bacteria and fungi). Examples thereof include cells derived from humans and cells derived from animals.

Examples of the cells derived from humans include HeLa, HL-60, HEK293, mesenchymal stem cells, hematopoietic stem cells, endothelial cells, cardiac muscle cells, osteoblasts, and hepatocytes.

Examples of the cells derived from animals include CHO, MDCK, and NIH3T3.

### <Antimicrobial Region>

The antimicrobial region is adjacent to the cell culture region.

The antimicrobial region is recessed with respect to the cell culture region.

The antimicrobial region has a plurality of fine projections that do not exceed the surface of the cell culture region in height. When the height of the fine projections does not exceed the surface of the cell culture region, this makes it possible to prevent cells attached to the surface of the cell culture region from contacting with the fine projections.

The depth of the antimicrobial region is not particularly limited and may be appropriately selected depending on the intended purpose. The depth thereof is, for example, 5 μm or more but 100 μm or less. Here, the depth of the antimicrobial region can be height from a bottom of the antimicrobial region to the surface of the cell culture region.

The depth is an average value and is, for example, an arithmetic mean value obtained from depths of 50 portions.

A shape of the fine projections is not particularly limited and may be appropriately selected depending on the intended purpose. Examples thereof include a cone shape and a pillar shape.

Examples of the cone include a circular cone and a pyramid. Examples of the pyramid include a trigonal pyramid and a quadrangular pyramid.

The shape, height, width, and intervals of the fine projections and the depth of the antimicrobial region can be confirmed, for example, by observing them with an electron microscope.

The height of the fine projections is not particularly limited and may be appropriately selected depending on the intended purpose, so long as the fine projections do not exceed the surface of the cell culture region. For example, the height of the fine projections may be 0.7 times or less, 0.5 times or less, or 0.2 times or less as high as the height from the bottom of the antimicrobial region to the surface of the cell culture region. However, being 0.7 times or less is preferable.

An upper limit of the height of the fine projections is not particularly limited and may be appropriately selected depending on the intended purpose. However, the height of the fine projections may be 0.05 times or more, or 0.1 times or more, as high as the height from the bottom of the antimicrobial region to the surface of the cell culture region.

Moreover, the height of the fine projections can be appropriately selected depending on a degree of a recession of the antimicrobial region with respect to the cell culture region. For example, the height of the fine projections may be 50 nm or more or may be 100 nm or more. In addition, the height of the fine projections may be 1,000 nm or less or may be 500 nm or less.

The height is an average value and is, for example, an arithmetic mean value obtained from height values of 50 fine projections.

An interval (pitch) of the plurality of fine projections in the antimicrobial region is not particularly limited and may be appropriately selected depending on the intended purpose. For example, the interval may be 50 nm or more, 100 nm or more, or 200 nm or more. In addition, for example, the interval may be 1,000 nm or less, 750 nm or less, or 500 nm or less.

The interval is an average value and is, for example, an arithmetic mean value obtained from intervals at 50 portions.

The width of the fine projections is not particularly limited and may be appropriately selected depending on the intended purpose. For example, the width may be 25 nm or more, 50 nm or more, or 100 nm or more. Moreover, for example, the width may be 1,000 nm or less, 600 nm or less, or 400 nm or less.

The width is an average value and is, for example, an arithmetic mean value obtained from widths of 50 fine projections.

The width is a width at the bottom of the fine projection.

For example, when the cell culture sheet is seen from a top surface thereof, a plurality of the antimicrobial regions are surrounded by the cell culture region.

In addition, when the cell culture sheet is seen from the top surface thereof, a plurality of the cell culture regions are surrounded by the antimicrobial region.

A ratio between the cell culture region and the antimicrobial region in the cell culture sheet is not particularly limited and may be appropriately selected depending on the intended purpose. For example, when the cell culture sheet is seen from a top surface thereof, an area ratio (cell culture region:antimicrobial region) may be 90:10 to 10:90, 80:20 to 20:80, 30:70 to 70:30, or 40:60 to 60:40.

Here, one example of a cell culture sheet will be described with reference to figures.

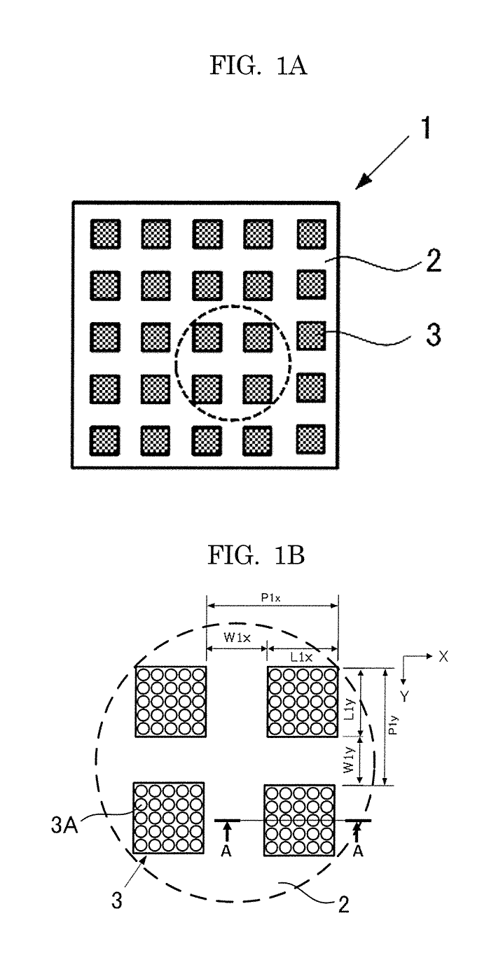

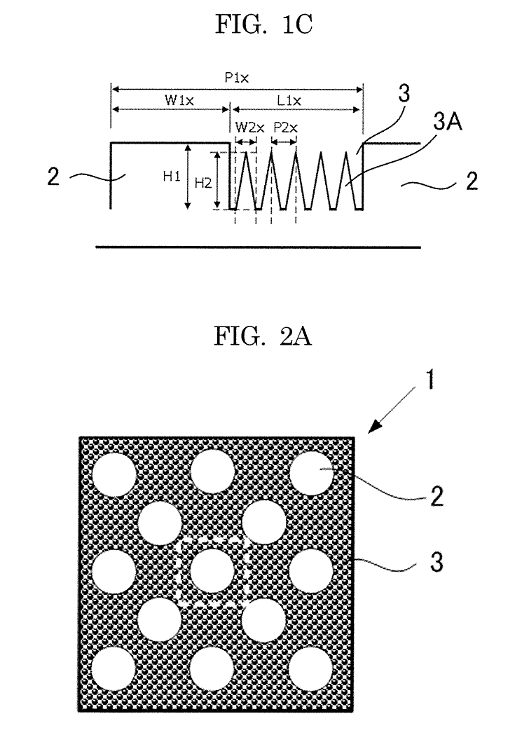

FIG. 1A to FIG. 1C are schematic views for presenting one example of a cell culture sheet.

When cell culture sheet 1 is seen from a top surface thereof, the cell culture sheet 1 presented in FIG. 1A to FIG. 1C has one continuous cell culture region 2 and a plurality of antimicrobial regions 3 surrounded by the one continuous cell culture region 2.

FIG. 1A is a top view of the cell culture sheet 1. FIG. 1B is an enlarged view of the portion circled by the dashed line in FIG. 1A. FIG. 1C is a cross-sectional view of AA in FIG. 1B.

The cell culture sheet 1 presented in FIG. 1A to FIG. 1C has one continuous cell culture region 2 and the plurality of antimicrobial regions 3, each of which is spaced from each other and is surrounded by the cell culture region 2. The antimicrobial regions 3 are recessed with respect to the cell culture region 2 (FIG. 1C).

Each antimicrobial region 3 includes a plurality of fine projections 3A within its region (FIG. 1B and FIG. 1C). A shape of the fine projection 3A has a circler cone shape (FIG. 1B and FIG. 1C).

In this example, a shape of the antimicrobial region 3 is square when the cell culture sheet 1 is seen from the top surface thereof. Widths (W1x and W1y) of the cell culture region 2 between the adjacent antimicrobial regions 3 are, for example, 5 μm or more but 100 μm or less. Width (L1x and L1y) of the antimicrobial region 3 is, for example, 5 μm or more but 100 μm or less. An interval (pitch: P1x=W1x+L1x, P1y=W1y+L1y) between the antimicrobial regions 3 is, for example, 10 μm or more but 200 μm or less.

The depth (H1) of the antimicrobial region 3 is, for example, 5 μm or more but 100 μm or less. The height (H2) of the fine projection 3A is, for example, 0.7 times or less as high as the depth (H1) of the antimicrobial region 3.

The width (W2x) of the fine projection 3A is, for example, 25 nm or more but 600 nm or less.

An interval (pitch: P2x) between the fine projections 3A is, for example, 50 nm or more but 1,000 nm or less.

In the cell culture sheet presented in FIG. 1A to FIG. 1C, cells intended to be cultured are placed and are cultured on the cell culture region 2, while cell walls of microorganisms intended to be antimicrobially treated are destroyed by the fine projections 3A in the antimicrobial regions 3 to express antimicrobial property.

Another one example of a cell culture sheet will be described with reference to figures.

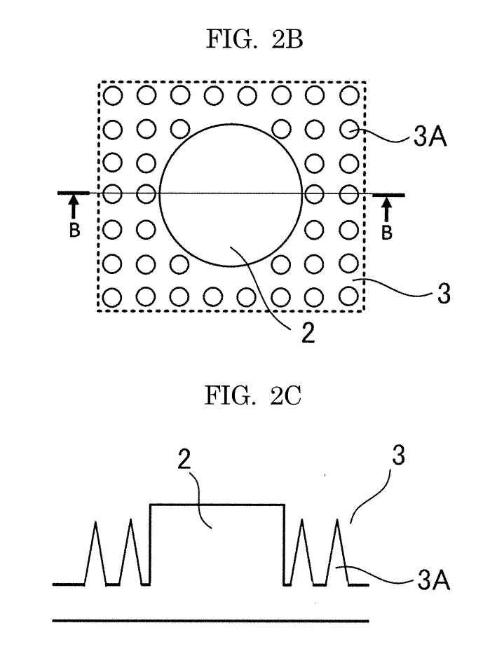

FIG. 2A to FIG. 2C are schematic views for presenting another one example of a cell culture sheet.

When cell culture sheet 1 is seen from a top surface thereof, the cell culture sheet 1 presented in FIG. 2A to FIG. 2C has a plurality of cell culture regions 2 and one continuous antimicrobial region 3 surrounded by the one continuous antimicrobial region 3.

FIG. 2A is a top view of the cell culture sheet 1. FIG. 2B is an enlarged view of the portion enclosed by the dashed line that is positioned at a center of FIG. 2A. FIG. 2C is a cross-sectional view of BB in FIG. 2B.

The cell culture sheet 1 presented in FIG. 2A to FIG. 2C has one continuous antimicrobial region 3 and the plurality of cell culture regions 2, each of which is spaced from each other and is surrounded by the antimicrobial region 3. The antimicrobial region 3 is recessed with respect to the cell culture regions 2 (FIG. 2C).

The antimicrobial region 3 has a plurality of fine projections 3A within its region (FIG. 2B and FIG. 2C). A shape of the fine projection 3A has a circler cone shape (FIG. 2B and FIG. 2C).

In this example, a shape of the cell culture region 2 is circular when the cell culture sheet 1 is seen from the top surface thereof. Width of the antimicrobial region 3 between the adjacent cell culture regions 2 is, for example, 5 μm or more but 100 μm or less. A diameter of the cell culture region 2 is, for example, 5 μm or more but 100 μm or less. An interval between the cell culture regions 2 is, for example, 10 μm or more but 200 μm or less.

The depth of the antimicrobial region 3 is, for example, 5 μm or more but 100 μm or less. The height of the fine projection 3A is, for example, 0.7 times or less as high as the depth of the antimicrobial region 3.

The width of the fine projection 3A is, for example, 25 nm or more but 600 nm or less.

An interval between the fine projections 3A is, for example, 50 nm or more but 1,000 nm or less.

In the cell culture sheet presented in FIG. 2A to FIG. 2C, cells intended to be cultured are placed and are cultured on the cell culture regions 2, while cell walls of microorganisms intended to be antimicrobially treated are destroyed by the fine projections 3A in the antimicrobial region 3 to express antimicrobial property.

Another one example of a cell culture sheet will be described with reference to figures.

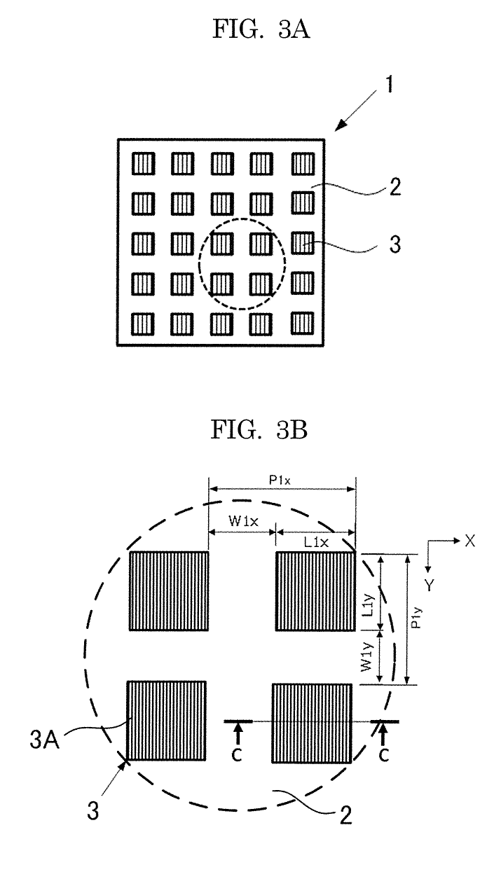

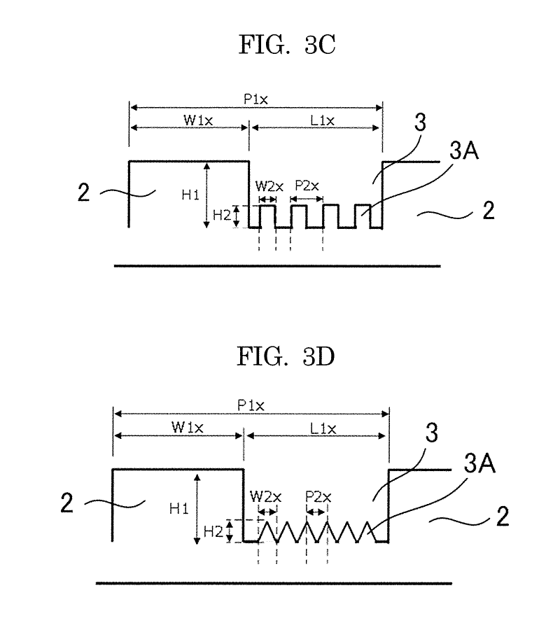

FIG. 3A to FIG. 3D are schematic views for presenting one example of a cell culture sheet.

When cell culture sheet 1 is seen from a top surface thereof, the cell culture sheet 1 presented in FIG. 3A to FIG. 3D has one continuous cell culture region 2 and a plurality of antimicrobial regions 3 surrounded by the one continuous cell culture region 2.

FIG. 3A is a top view of the cell culture sheet 1. FIG. 3B is an enlarged view of the portion circled by the dashed line in FIG. 3A. FIG. 3C is one example of a cross-sectional view of CC in FIG. 3B. FIG. 3D is also a cross-sectional view of CC in FIG. 3B. FIG. 3D is one example of the fine projections 3A having a different shape from that of FIG. 3C.

The cell culture sheet 1 presented in FIG. 3A to FIG. 3D has one continuous cell culture region 2 and the plurality of antimicrobial regions 3, each of which is spaced from each other and is surrounded by the cell culture region 2. The antimicrobial regions 3 are recessed with respect to the cell culture region 2 (FIG. 3C and FIG. 3D).

In this example, a shape of the antimicrobial region 3 is square when the cell culture sheet 1 is seen from the top surface thereof.

The antimicrobial regions 3 have a plurality of fine projections 3A within their region (FIG. 3B, FIG. 3C, and FIG. 3D). In this example, when the cell culture sheet 1 is seen from the top surface thereof, the plurality of fine projections 3A, each of which has such a rectangular shape that extends from one side to the opposite side across the antimicrobial region 3, are provided at fixed intervals within the antimicrobial region 3 (FIG. 3B). When the fine projection 3A is seen at a cross section of CC, one example of the cross section of the fine projection 3A is rectangular (FIG. 3C), and another example of the cross section of the fine projection 3A is triangle (FIG. 3D).

Widths (W1x and W1y) of the cell culture region 2 between the adjacent antimicrobial regions 3 are, for example, 5 μm or more but 100 μm or less. Widths (L1x and L1y) between the antimicrobial regions 3 are, for example, 5 μm or more but 100 μm or less. An interval (pitch: P1x=W1x+L1x, P1y=W1y+L1y) between the antimicrobial regions 3 is, for example, 10 μm or more but 200 μm or less.

The depth (H1) of the antimicrobial region 3 is, for example, 5 μm or more but 100 μm or less. The height (H2) of the fine projection 3A is, for example, 0.7 times or less as high as the depth (H1) of the antimicrobial region 3.

The width (W2x) of the fine projection 3A is, for example, 25 nm or more but 600 nm or less.

An interval (pitch: P2x) between the fine projections 3A is, for example, 50 nm or more but 1,000 nm or less.

In the cell culture sheets presented in FIG. 3A to FIG. 3D, cells intended to be cultured are placed and are cultured on the cell culture region 2. On the other hand, the height (H2) of the fine projection 3A is much smaller than the depth (H1) of the antimicrobial region 3, and therefore microorganisms intended to be antimicrobially treated will remain at a deep position and between the fine projections 3A within the antimicrobial region 3. This prevents microorganisms from colonization and formation of biofilms, and thus antimicrobial property with respect to the microorganisms is expressed.

### <Base Material>

A material of the base material is not particularly limited and may be appropriately selected depending on the intended purpose. Examples thereof include glasses and resins.

Examples of the resins include polyethylene terephthalate.

### <Culture Layer>

A material of the culture layer is not particularly limited and may be appropriately selected depending on the intended purpose, so long as it has no toxicity to the cells. Examples thereof include thermoplastic resins and cured products of curable organic materials.

Examples of the thermoplastic resins include polyolefin-based homopolymers (e.g., polypropylene and polyethylene), polyolefin-based copolymers (e.g., polyvinyl acetate and polyvinyl alcohol), cycloolefin-based polymers (e.g., cycloolefin polymer and cycloolefin copolymer), polystyrene, polyesters (e.g., polyethylene terephthalate), polyurethane, polyamide, polyvinyl chloride, polymethyl methacrylate, and polycarbonate.

Examples of the cured products of curable organic materials include cured products of active-energy-ray-curable organic materials and cured products of thermosetting organic materials. Here, the curable organic materials are generally referred to as a “curable resin” including materials having a low molecular weight.

Examples of the active energy rays include ultraviolet rays and electron beams.

### <Method for Producing Cell Culture Sheet>

A method for producing the cell culture sheet is not particularly limited and may be appropriately selected depending on the intended purpose. Examples thereof include a method for producing the cell culture sheet by forming, on a base material, a culture layer including the cell culture region and the antimicrobial region.

One exemplary method for forming the culture layer is, for example, a method for forming the culture layer including the cell culture region and the antimicrobial region, by charging a melted precursor of the culture layer into an original mold or a stamper engraved to be capable of forming structures of the cell culture region and the antimicrobial region, and then curing the precursor.

As one exemplary method for producing the cell culture sheet, a production process using an ultraviolet-ray-curable resin will be described below.

First, an original mold having surface structures on which the cell culture region and the antimicrobial region have been inverted is produced. Examples of the original mold include general materials such as metals (e.g., iron-based metals, aluminum-based metals, and copper-based metals), glasses, and silicones. As a processing method for forming the surface structures on the original mold, it is possible to use known approaches such as mechanical cutting, laser lithography, interference lithography using laser, electron beam lithography, and etching.

Next, an acrylic ultraviolet-ray-curable resin (a precursor of the culture layer) is added dropwise and is spread through coating on a PET film as a base material.

Then, the original mold and the PET film are pressed through, for example, a rubber roller, and the ultraviolet-ray-curable resin is sufficiently attached to the fine surface structure that are engraved in the original mold.

Then, ultraviolet rays having a desired wavelength are emitted from the rear surface of the PET film and the ultraviolet-ray-curable resin is cured to form a culture layer.

Finally, the PET film on which the culture layer is stacked is peeled from the original mold to obtain a cell culture sheet.

### EXAMPLES

The present invention will be described with reference to the following Examples. However, it should not be construed that the present invention is not limited to these Examples.

### Example 1

### <Preparation of Cell Culture Sheet>

The following materials were used to prepare a cell culture sheet.

### [Material]

Base material: a PET film with an easily adhesive layer (available from TOYOBO CO., LTD., product name: COSMOSHINE A4300, thickness: 0.18 mm)

Resin composition:

Ultraviolet-ray-curable resin (available from kyoeisha Chemical Co., Ltd., product name: LIGHT ESTER HO-250 (N), 94 wt %)

Photoinitiator (available from BASF, product name: IRGACURE 127, 6 wt %)

A resin composition was placed on a base material. Then, in order to form a cell culture sheet presented in FIG. 1A to FIG. 1C, a transferable original mold capable of forming a shape presented in the following Table 1 was used to transfer the resin composition so as to have concave and convex structures. Then, the resin was subjected to photocuring under the following photocuring conditions to obtain a cell culture sheet.

### [Photocuring Conditions]

Light source: metal halide λ365 nm

Radiation dose: 1,081 mJ/cm²

TABLE 1

| W1x | L1x | P1x | W1y | L1y | P1y | H1 | H2 | W2x | P2x | Fine |
|---|---|---|---|---|---|---|---|---|---|---|
| μm | μm | μm | μm | μm | μm | μm | nm | nm | nm | projections |
| 15 | 15 | 30 | 15 | 15 | 30 | 15 | 230 | 150 | 230 | Circular cone |

Here, the fine projections have a circular cone shape and are arranged in hexagonal pattern.

When the cell culture sheet obtained is seen from a top surface thereof, a plurality of antimicrobial regions are surrounded by one continuous cell culture region.

### Comparative Example 1

### <Preparation of Cell Culture Sheet>

The same materials as those used in Example 1 were used to prepare a cell culture sheet with the whole surface being flat.

A resin composition was placed on a base material, a cycloolefin resin film (available from Zeon Corporation, product name: ZF14-060, thickness: 0.06 mm) was pasted on the resin composition. Then, the resin was subjected to photocuring via the film under the following photocuring conditions to obtain the cell culture sheet.

### [Photocuring Conditions]

Light source: metal halide λ365 nm

Radiation dose: 1,081 mJ/cm²

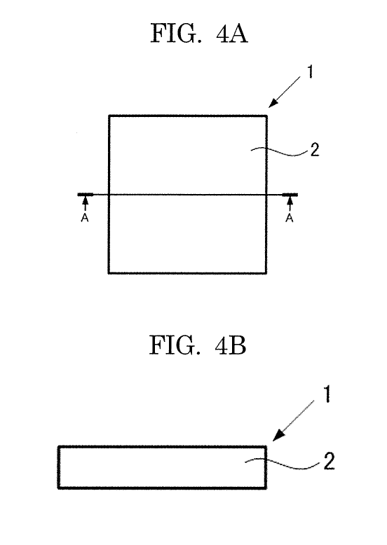

Schematic views of the cell culture sheet obtained are presented in FIG. 4A and FIG. 4B.

FIG. 4A is a top view thereof and FIG. 4B is a cross-sectional view of AA in FIG. 4A.

The surface of cell culture sheet 1 is formed of flat cell culture region 2.

### [Antimicrobial Test]

An antimicrobial test was performed according to JISZ 2801 “treated antimicrobe product-antimicrobial test method⋅antimicrobial effects”. Staphylococcus aureus (NBRC 12732) was used as a bacterial strain.

Test pieces (5 cm×5 cm) of the cell culture sheets of Example 1 and Comparative Example 1 were each placed on a dish. Then, the test bacterial liquid (0.4 ml) was added dropwise and the dish was covered with a polyethylene film (4 cm×4 cm). The dish was incubated at 35° C. and 90% RH for 24 hours. Each test piece was removed and was charged into a Stomachere bag. Then, 10 ml of an SCDLP medium was added thereto and the test bacteria were washed out. The number of the bacteria in the liquid containing the washed-out bacteria was measured through the agar plate medium method to calculate an antimicrobial activity value.

### <<Evaluation>>

The viable cell count is the number of bacteria per 1 cm² and the values presented in Table 2 were each an average value obtained from the values of three tests. The antimicrobial activity value is a difference between an average value of logarithmic values of viable cell counts on a non-treated film (made of polyethylene) and an average value of logarithmic values of viable cell counts on the above-described treated product. Generally, it can be judged that the antimicrobial activity value of 2.0 or more exhibits an antimicrobial property.

TABLE 2

| | | | | | | | | | | | | Viable | |
|---|---|---|---|---|---|---|---|---|---|---|---|---|---|
| | W1x | L1x | P1x | W1y | L1y | P1y | H1 | H2 | W2x | P2x | Fine | cell count | Antimicrobial |
| | μm | μm | μm | μm | μm | μm | μm | nm | nm | nm | projections | (/cm²) | activity value |
| Ex. 1 | 14.8 | 14.5 | 29.3 | 14.9 | 14.4 | 29.3 | 14.7 | 192 | 153 | 244 | Circular cone | 6.6 × 10² | 3.5 |

| | | | | |
|---|---|---|---|---|
| Comp. | — | None | 4.9 × 10⁴ | 1.4 |
| Ex. 1 | | | | |

From Table 2, it is found that Example of the present invention exhibits an antimicrobial activity value of 2.0 or more and exhibits antimicrobial effects.

### INDUSTRIAL APPLICABILITY

The cell culture sheet of the present invention exhibits an effective antimicrobial action on microorganisms and thus can be used for culturing cells without using an antibiotic agent and an antimicrobial agent.

What is claimed is:

1. A cell culture sheet comprising:
at least one cell culture region contributing to cell culture; and
at least one antimicrobial region exhibiting antimicrobial action, which is adjacent to the cell culture region, recessed with respect to the cell culture region, and has a plurality of fine projections that do not exceed a surface of the cell culture region in height.

2. The cell culture sheet according to claim 1, wherein the fine projections have a cone shape or a pillar shape.

3. The cell culture sheet according to claim 1, wherein when the cell culture sheet is seen from a top surface thereof, a plurality of the antimicrobial regions are surrounded by the cell culture region.

4. The cell culture sheet according to claim 1, wherein when the cell culture sheet is seen from a top surface thereof, a plurality of the cell culture regions are surrounded by the antimicrobial region.

5. The cell culture sheet according to claim 1, wherein height of the fine projections is 0.7 times or less as high as height from a bottom of the antimicrobial region to the surface of the cell culture region.

6. The cell culture sheet according to claim 1, wherein depth of the antimicrobial region is 5 μm or more but 100 μm or less.

7. The cell culture sheet according to claim 1, wherein the cell culture sheet comprises a base material and a culture layer on the base material, the culture layer including the cell culture region and the antimicrobial region.
